(19) United States
(12) Patent Application Publication
(10) Pub. No.: US 2022/0287694 A1
(43) Pub. Date: Sep. 15, 2022

(54) Comfortable Patient Collected Home PAP

(71) Applicant: Jovanni Neblett-Blackmon, Acworth, GA (US)

(72) Inventor: Jovanni Neblett-Blackmon, Acworth, GA (US)

(21) Appl. No.: 17/195,655

(22) Filed: Mar. 9, 2021

(51) Int. Cl. A61B 10/02

(52) U.S. Cl. CPC A61B 10/0291; A61B 2010/0216

(57) ABSTRACT

The present invention relates to a device for collection of tissue cells from a cervical face and endocervical canal of a uterus which material to ensure elasticity, adhesion of cells to the surface for collection, and comfort for the patient. The assembly provides elastic material to affix the cell collection surface to the rod with bristles rather than traditional rigid mechanical clamps, use of three distinct types of bristles specialized for collecting cells from each of the cervical os, use of a single elastic material for the brush bristles and for affixing the cell collection surface to the shaft/rod. The assembly also ensures protection of the collected cell specimens from contamination by inversion of the elastic bristle-bearing material prior to removal from the vagina.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The present invention relates generally to the field for safe collection of specimens. More particularly, the present invention relates to medical devices and procedures, and in particular, to medical devices and procedures for conducting a Pap smear test.

### DESCRIPTION OF THE RELATED ART

The Papanicolaou test (also called Pap smear, Pap test, cervical smear, or smear test) is a screening test used to detect potentially pre-cancerous and cancerous processes in the endocervical canal (transformation zone) of the female reproductive system.

A Pap smear is accomplished by usage of a cell extraction device such as a Pap smear spatula and Pap smear brush or alternatively by utilization of a Pap smear broom. In either case, the medical practitioner begins by inserting a speculum into the woman's vagina, which spreads the vagina open and allows access to the cervix. If using the spatula and brush, the medical practitioner then collects a sample of cells from the outer opening of the cervix by scraping it with the spatula. Then, an endocervical brush is rotated in the central opening of the cervix if a better sample is needed. Alternatively, the medical practitioner may opt to use a plastic-fronded broom (Pap smear broom) in place of the spatula and brush. The cells are removed from the patient and are placed on a glass slide or in a vial of solution that is sent to a laboratory to be checked for abnormalities.

### Speculum Problems

As stated above, the prior art method of conducting a Pap smear involves the use of a speculum to spread open the vagina. The utilization of the speculum can be extremely uncomfortable and painful to many women as the vagina is spread apart. Some women have even described the speculum as agonizing. The fear and apprehension associated with the speculum has unfortunately caused many women to delay the Pap smear test or, in some cases, to even avoid it entirely. This is unacceptable because it can allow preventable cancer to remain undiagnosed.

What is needed is a better device and method for conducting a Pap smear which is easier and safe to use. It has to be noted that the current invention proposes an advancement where the it provides a new approach of conducting PAP test. A safe and retractable bio-molecular sampling and transportation system in accordance with the present invention utilizes a unique mechanism.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The present invention seeks to improve devices for the safe collection of specimens especially for PAP test at home without the need of going to physician.

Another object of the present invention is to provide a device which uses elastic material to affix the cell collection to its projection surfaces (not rigid mechanical clamps as others have used).

It is a more particular object of the present invention to provide an improved system where it uses two distinct types of bristles on the head of the device specialized for collecting cells from each aspect of the transformation zone; the ectocervix and the endocervix (inside the cervical os)(longer & thinner) or the vaginal wall (thicker & shorter).

It is also the objective of invention to provide use of a single elastic material for the brush bristles, which allow for affixing the cell collection surface to the silicon shealth. All others mount the bristles (made of one material) onto something made of another material.

It is also object of the invention to provide an assembly where the purpose of this instrument is to provide an instrument that gives the patient more control for comfort and self administering.

It is further the objective of the invention to provide assembly which is aimed for protection of the collected cell specimens from contamination by inversion of the elastic bristle-bearing material prior to removal from the vagina.

It is also the objective of the invention to provide an assembly which increases control over the surgical site and enhances both the speed and quality of the entire procedure.

It is also the objective of the invention to provide a whole construction which is compatible with high volume manufacturing methods for all of the components. The low manufacturing cost means that this high-quality portable instrument may be made available to hospitals and clinics that may otherwise not be able to afford a quality instrument. It also makes the assembly highly applicable to the developing world and exposes a wider public audience to first hand scientific advancements.

Thus, it is the objective to provide a new and improved form of an advancement assisting the process of PAP test. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

FIG. 1 discloses the view of PAP device as per preferred embodiments of the invention.

FIG. 2 discloses the exploded view of PAP device as per preferred embodiments of the invention.

FIG. 3 discloses the flexible axial view of invention as per preferred embodiments of the invention.

FIG. 4 discloses the insertion point of assembly as per preferred embodiments of the invention.

FIG. 5 discloses the retraction point of assembly as per preferred embodiments of the invention.

FIG. 6 discloses the rotational view of assembly as per preferred embodiments of the invention.

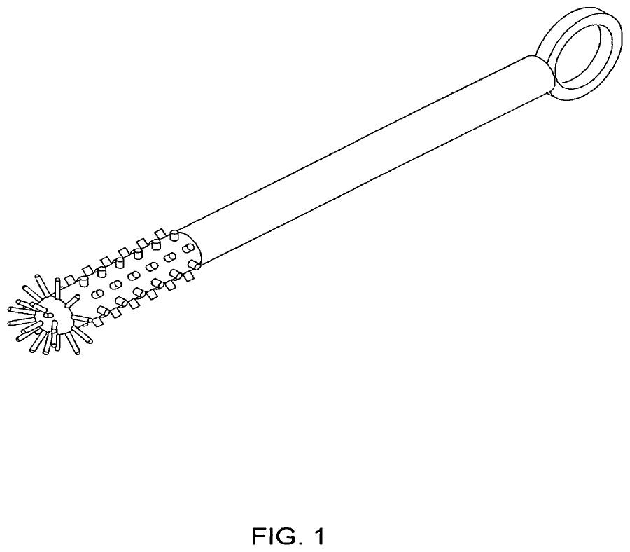

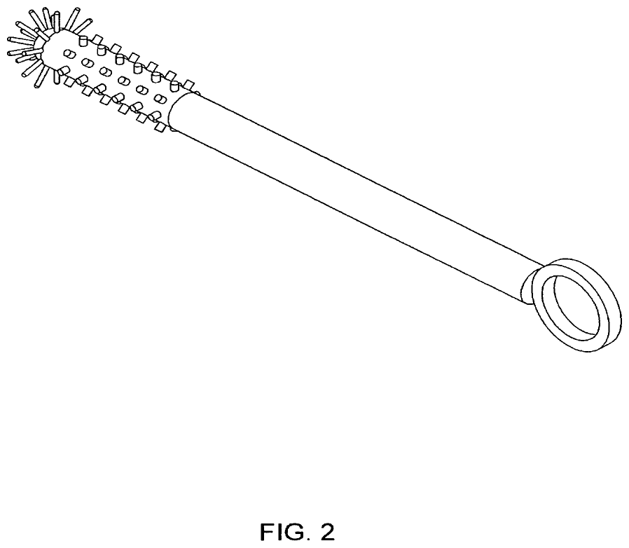

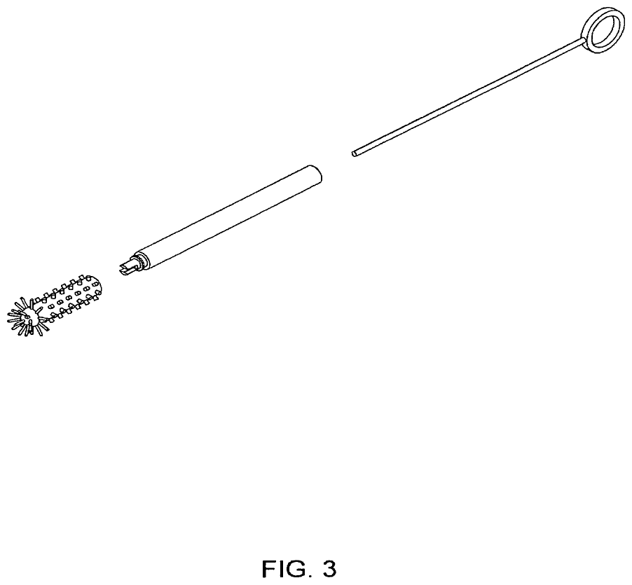

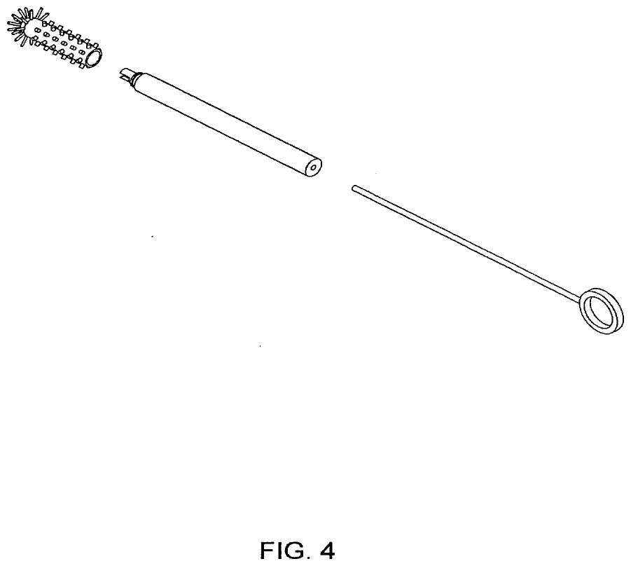

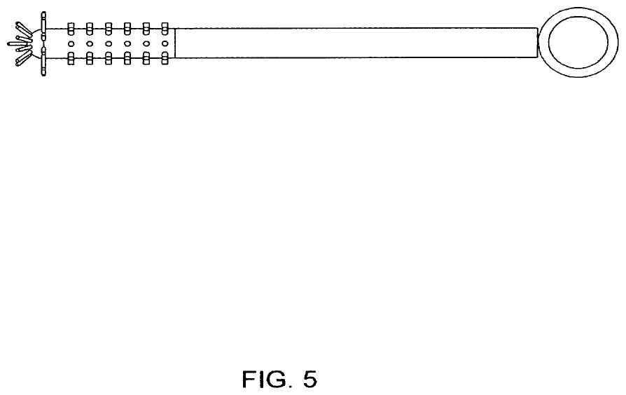

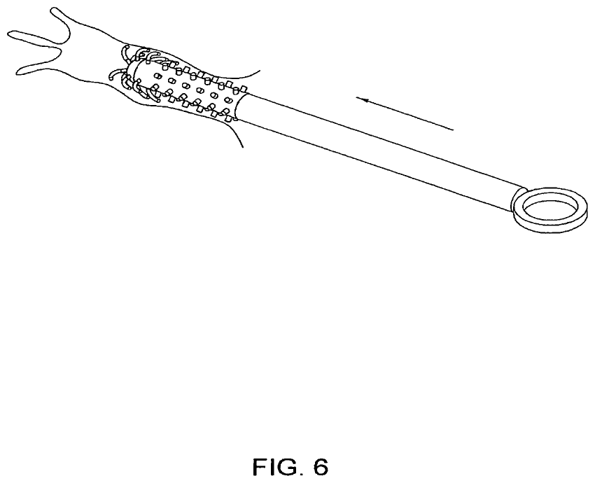

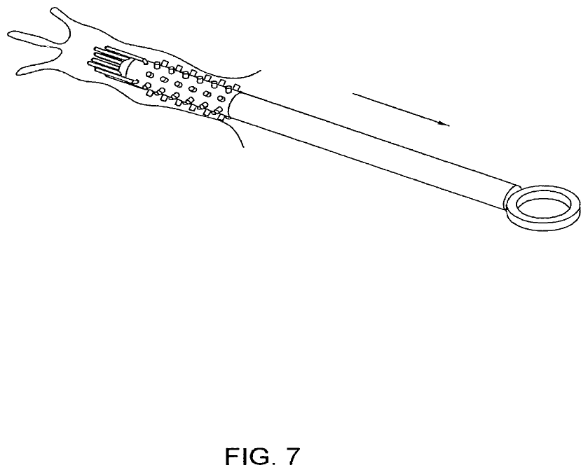

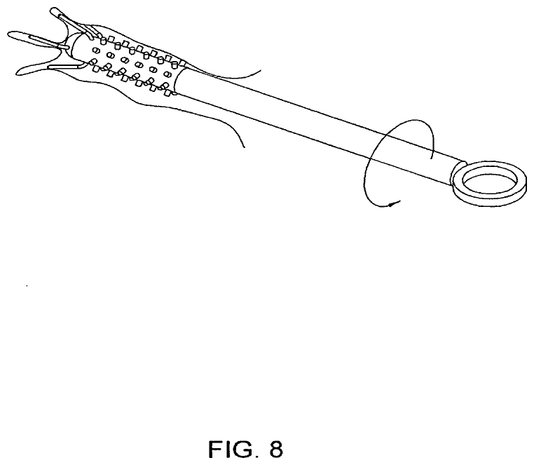

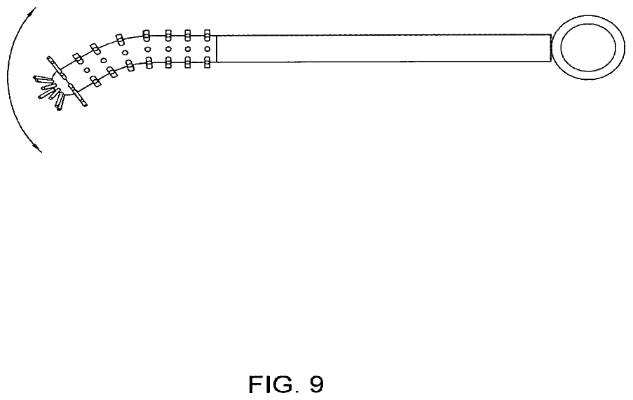

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Over the last 40 years, the death rate from cervical cancer has dropped dramatically due to the widespread use of the Papanicolaou test. This test involves the collection of cells from the cervical face, the endocervical canal and occasionally from the vaginal wall.

These cells are then microscopically examined for evidence of precancerous or cancerous growth. The periodic use of this test permits early detection of malignancy so that preventive therapy can be initiated at a time when it will yield the highest possible success rate.

For many years the standard method for collection of these cells has been a by a physician in the office with the use of a metal speculum and a plastic spatula or plastic cervical broom for the cervical cells. A variety of slightly different shapes have been used in an effort to allow the clinician to collect cells from the cervical face as well as the endocervical canal.

For the most part, these designs have been able to retrieve cells from the surface of the cervix and the vaginal wall but have frequently failed to collect adequate cell samples from the endocervical canal. This has been a serious problem because any sample which does not include endocervical cells is deemed to be inadequate for proper | WO 93/1 708 PCT/US92/10234 2 interpretation and diagnosis. Further, many women who have had an inadequate sample taken are reluctant to have the procedure repeated and may be placed at risk for progression of their disease until their next periodic checkup.

A delay in the diagnosis of cervical or uterine cancer can result in the need for far more radical treatment procedures than would be needed if the cancer were detected earlier. In the worst case, a delay in diagnosis could result in the progression from treatable cancer to a terminal cancer.

Recent modifications in collection procedures have added a cotton swab to the technique in an effort to collect endocervical cells or HPV samples. The cotton swab is inserted into the cervical canal and rotated. Although the cotton swab may somewhat improve the yield, it is not abrasive enough to scrape the endocervical canal and consistently retrieve an adequate sample.

Another modification made in an attempt to collect endocervical cells is what is called a “cytology brush.” This cytology brush has on the end of a long handle a conically-tapering brush made of spiraling bristles. When the brush is rotated within the endocervical canal, cells become entrapped by the brush. However, while the cytology brush can consistently obtain endocervical and ectocervical cells, the brush is too abrasive, and so its use increases the incidence of bleeding and discomfort and, in addition, adds considerable cost to a very price-sensitive procedure.

If bleeding occurs during collection, the red blood cells may obscure other cells on the cytology slide and make interpretation difficult or impossible. When this happens, the cell collection procedure must be repeated.

According to one aspect of the present invention there is provided a device for collection of tissue cells from a cervical face and endocervical canal of a cervix comprising: an elongated handle having a forward end and a rear end; tissue sampling means, located at the forward end of the said handle, for simultaneously collecting tissue cells from the cervical face and endocervical canal, said sampling means comprising: first means providing a plurality of bristles oriented in a row and in a plane for contacting and collecting cells within the endocervical canal as said handle is rotated, said first means aligned with said handle and extending from said forward end thereof; and (ii) second means providing a plurality of bristles oriented in a row and in the same said plane for contacting and collecting cells from the cervical face as said handle is rotated, said second means extending from said forward end of said handle in a direction generally orthogonal to said first means; and flexible means positioned at said 0° forward end of said handle for bending said tissue oo ‘sampling means at an acute angle with respect to the handle for assistance in dislodging the collected tissue cells from said first and second means onto an area for examination.

When the device is inserted into the vagina, the longitudinally extending bristles insert easily into the endocervical canal. and the second set of bristles on the top of the device make contact with the ectocervical surface. Simultaneously, the second set of bristles on the top of the device laterally press against the cervical face and make contact with the ectocervix sufficient forward pressure to flex the second set of bristles slightly and collect cells. These are cells from the transformation zone (which equals the endocervical and ectocervical cells), which are needed for an adequate sample. The third set of bristles take a sample from the vagina wall that can be used for HPV testing.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1: A device for collection of tissue cells from a cervical face and endocervical canal of a uterus comprising: an elongated handle having a forward end and a rear end; tissue sampling means, located at the forward end of said handle, for simultaneously collecting tissue cells from the cervical face and endocervical canal, said sampling means comprising: first means providing a plurality of bristles oriented in a row and in a plane for contacting and collecting cells within the endocervical canal as said handle is rotated, said first means aligned with said handle and extending from said forward end thereof;
A device as per claim 1, where use of elastic material to affix the cell collection surface to the bristles (not rigid mechanical clamps as others have used) and the inherent elasticity of the material to grip the rod/shaft;
A device as per claim 1, where use of three distinct types of bristles specialized for collecting cells from each of the cervical os (longer & thinner, one that goes into endocervical canal and second on the ectocervix) and the vaginal wall bristles (thicker & shorter);
A device as per claim 1, where use of a single elastic material for the brush bristles and for affixing the cell collection surface to the shaft/rod. All others mount the bristles (made of one material) onto something made of another material;
A device as per claim 1, where protection of the collected cell specimens from contamination by inversion of the elastic bristle-bearing material prior to removal from the vagina; and
A device as per claim 1, where use of a material combining three properties elasticity, adhesion of cells to the surface for collection, and comfort for the patient is proposed.
